(19) United States
(12) Patent Application Publication
(10) Pub. No.: US 2026/0240787 A1
(43) Pub. Date: Aug. 20, 2026

(54) PROTEIN NANOPARTICLES TO INCREASE EXERCISE ENDURANCE, FUNCTIONALITY AND LONGEVITY IN CANCER AND NON-CANCER PATIENTS

(71) Applicant: Richard C.K. Yen, Yorba Linda, CA (US)

(72) Inventor: Richard C.K. Yen, Yorba Linda, CA (US)

(21) Appl. No.: 19/554,251

(22) Filed: Mar. 2, 2026

Related U.S. Application Data
(63) Continuation-in-part of application No. 19/410,002, filed on Dec. 5, 2025.
(63) Continuation-in-part of application No. 19/409,987, filed on Dec. 5, 2025.
(63) Continuation-in-part of application No. 19/190,243, filed on Apr. 25, 2025.
(63) Continuation-in-part of application No. 18/735,523, filed on Jun. 6, 2024.
(63) Continuation-in-part of application No. 18/295,829, filed on Apr. 4, 2023.
(63) Continuation-in-part of application No. 17/208,736, filed on Mar. 22, 2021, now abandoned.
(60) Provisional application No. 63/834,196, filed on Mar. 3, 2025.

(51) Int. Cl. A61K 9/51; A61K 9/00; A61K 31/706

(52) U.S. Cl. CPC A61K 9/5169; A61K 9/0019; A61K 9/5192; A61K 31/706

(57) ABSTRACT

This invention discloses the beneficial effects of the intravenous administration of protein nanospheres particularly Fibrinogen-coated Albumin Spheres, including the mobilization of stem cells from the bone marrow or other sites, leading to an effective build-up of muscle mass and/or repair of other dysfunctional conditions. The benefits can be produced in the healthy individual, regardless of age, which can lead to greater muscle mass, better exercise endurance and stronger performance, resulting in increased health and even life span. The invention can potentially be applied to mitigate the harm expected in space travel and habitation, including the effects of microgravity and cosmic radiation. In cancer patients with pre-cancer-treatment impairments, FPS can improve deficient domains such as poor physical performance, cognitive, nutritional and psychological deficiencies before and after cancer treatment.

### CROSS-REFERENCE TO RELATED APPLICATION

This application claims the benefit of priority under 35 U.S.C. § 119 (e) based upon co-pending U.S. provisional patent application Ser. No. US 63/834,196 filed on Mar. 3, 2025. The entire disclosure of the prior provisional application is incorporated herein by reference. This application is a continuation-in-part under 35 U.S.C. § 120 based upon co-pending: U.S. patent application Ser. No. 19/410,002 filed on Dec. 5, 2025; U.S. patent application Ser. No. 19/409,987 filed on Dec. 5, 2025; U.S. patent application Ser. No. 19/190,246 filed on Apr. 25, 2025; U.S. patent application Ser. No. 18/812,320 filed on Aug. 22, 2024; U.S. patent application Ser. No. 18/735,523 filed on Jun. 3, 2024; U.S. patent application Ser. No. 18/295,829 filed on Apr. 4, 2023; and U.S. patent application Ser. No. 17/208,736 filed on Mar. 22, 2021, which are incorporated herein by reference in its entirety.

### BACKGROUND

### Technical Field

This invention is in the field of regenerative biology, longevity medicine and improvements in cancer survival. It concerns the administration of protein nanoparticles intravenously to enrich stem cells and pluripotent cells including those residing in the bone marrow and those that are already in the muscles, with the result that the individual, regardless of age or cancer status, can exhibit greater exercise endurance, be healthier and live longer; and in the case of cancer patients gain better survival outcomes.

In some aspects, the present technology relates to a protein nanoparticles to increase exercise endurance, functionality and longevity in cancer and non-cancer patients for use in connection with enriching stem cells and pluripotent cells by intravenously administration of protein nanoparticles.

### Background Description

Pluripotent cells are cells that have the capacity to differentiate from their “primitive state” into mature functional cells which form the tissues and organs of the body. They are often called stem cells; and historically stem cells are recognized as being located inside the bone marrow. These stem cells develop into various “lineages” which are the various stages of maturation which will eventually form fully differentiated cells which constitute the various cellular components of the body. However, recent research revealed that some “stem cells” can be located in other tissues, such as the endothelium—these cells are known to differentiate also, e.g. into bone tissues; and therefore, these cells are also called “progenitor cells.” In addition, some cells can be flushed from various organs (e.g. kidneys) which are called “perivascular stromal cells” which can also be useful as regenerative cells (“Clinical-Grade Isolated Human Kidney Perivascular Stromal Cells as an Organotypic Cell Source for Kidney Regenerative Medicine” authored by Leuning et al., at Stem Cells Transl Med. 2017 February; 6 (2): 405-418.) In addition, pluripotent cells can be isolated from many other tissues, such as the muscle or the adipose tissues. Therefore, regardless of their different location of origin, in this disclosure all these “regenerative” cells as a group will be called pluripotent cells (even though some may be totipotent), or stem cells or progenitor cells.

For patients who have specific diseases that may be mitigated by stem cell therapy, there are two main methods of getting these regenerative “donor” cells:
(a) by harvesting the patient's own stem cells or by induction of some mature cells obtained from the patient (i.e. reverse-conversion of certain mature cells into less mature cells which can then differentiate into the “wanted” cell types; and
(b) by the administration of pluripotent cells obtained from sources other than the patient himself, e.g. umbilical cord cells or bone marrow cells obtained from a compatible donor.

By definition, all these harvested (primitive) cells are used directly for transfusion into the patient, or the cells (if they are at a stage more differentiated than the totipotent stem cells) need to undergo certain “processes” in vitro (to be called “processed cells” in this disclosure) to become more useful for the specific medical indications; e.g. (a) by harvesting the stem cells and then directly enriching their number or concentration in vitro, or (b) by an indirect approach, e.g. inducing certain mature cells harvested from the patient into (less mature) pluripotent cells (“initial processed cells”), and then further inducing these “initial processed cells” towards a particular pathway (“desired processed cells”.) The main purpose of trying to channel the processed cells into a particular pathway is to (i) obtain enough quantity of the wanted pluripotent donor cells; and (ii) avoid the formation of “unwanted” cells from these “initial processed cells” which can develop into tumor cells (instead of, or in addition to the desired cell type.) These steps are complicated and expensive, not to mention that there is no guarantee that the “desired processed cells” even if they are obtained, will perform well without side effects.

In terms of increasing exercise tolerance, the prior arts are full of advice on how to exercise well to build up muscle mass. One specific article disclosed the importance of muscle-derived stem cells (MDSC): “The role and therapeutic potential of stem cells in skeletal muscle in sarcopenia” by Liu et al, in Stem Cell Research & Therapy 13, article number 28 (2022). The authors stated in their introduction that “Muscle-derived stem cells (MDSCs) and satellite cells (SCs) are the two most commonly studied stem cell populations from skeletal muscle. To date, considerable progress has been achieved in understanding the complex associations between stem cells in muscle and the occurrence and treatment of sarcopenia.” However, there is no discussion on the role of protein nanoparticles in stimulating the production of MDSCs or SCs. Therefore, the administration of protein nanoparticles to increase exercise endurance and to increase the patient's ability to enjoy a healthy old age, with the result of living longer lives, is novel and non-obvious from disclosures in the prior arts.

### SUMMARY

In view of the foregoing disadvantages inherent in the known types of Stem cell mobilization at least some embodiments of the present technology provides a novel protein nanoparticles to increase exercise endurance, functionality and longevity in cancer and non-cancer patients, and overcomes one or more of the mentioned disadvantages and drawbacks of the prior art. As such, the general purpose of at least some embodiments of the present technology, which will be described subsequently in greater detail, is to provide a new and novel protein nanoparticles to increase exercise endurance, functionality and longevity in cancer and non-cancer patients which has all the advantages of the prior art mentioned herein and many novel features that result in a protein nanoparticles to increase exercise endurance, functionality and longevity in cancer and non-cancer patients which is not anticipated, rendered obvious, suggested, or even implied by the prior art, either alone or in any combination thereof.

According to one aspect, the present technology can include an albumin nanoparticle suspension containing submicron albumin spheres configured for effecting muscle-derived stem cells (MDSCs) or satellite cells (SCs) in a muscle of a subject in need thereof. A therapeutically effective amount of the albumin nanoparticle suspension being administered intravenously to the subject, and the albumin spheres being configured to suppress pro-inflammatory molecules.

According to another aspect, the present technology can include a method of effecting MDSCs or SCs in a muscle of a subject in need thereof using an albumin nanoparticle suspension containing submicron albumin spheres. The method can include the step of administering intravenously a therapeutically effective amount of the albumin nanoparticle suspension containing the submicron albumin spheres to the subject. The albumin spheres being configured to suppress pro-inflammatory molecules.

According to yet another aspect, the present technology can include a method of using an albumin nanoparticle suspension containing submicron albumin spheres to increase MDSCs or SCs in a muscle of a subject in need thereof. The method can include the steps of providing a suspension including fibrinogen-coated albumin nanospheres prepared by coating blank albumin spheres with a solution containing human fibrinogen. Then administering intravenously a therapeutically effective amount of the albumin nanoparticle suspension to the subject. Increasing a number of the MDSCs or the SCs in the muscle of the subject by promoting in vivo mobilization of stem cells to the muscle to increase muscle mass.

In some embodiments, the albumin spheres of the albumin nanoparticle suspension can be bound with fibrinogen molecules to produce Fibrinogen Albumin Spheres (FAS).

In some embodiments, the effect to the MDSCs or the SCs can be an increase in muscle mass in the subject.

In some embodiments, the effect to the MDSCs or the SCs can be a mitigation of microgravity or space radiation-induced damage to the muscle of the subject.

In some embodiments, the effect to the MDSCs or the SCs is to CD34+ cells can reside in the muscle of the subject.

Some embodiments of the present technology can include a step of increasing a number of the MDSCs or the SCs in the muscle of the subject by promoting in vivo mobilization of stem cells to the muscle.

In some embodiments, the stem cells can originate from a location not the muscle.

In some embodiments, the stem cells can originate from bone marrow or from endothelial cells.

Some embodiments of the present technology can include a step of administering to the subject a therapeutically effective amount of one or more co-factor precursors.

In some embodiments, the co-factor precursors can be selected from the group consisting of Nicotinamide Riboside (NR), and Nicotinamide MonoNucleotide (NMN).

In some embodiments, the increase to the number of the MDSCs or the SCs can provide a mitigation of microgravity or space radiation-induced damage to the muscle of the subject.

In some embodiments, the increase to the number of the MDSCs or the SCs can be to CD34+ cells residing in the muscle of the subject.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods and systems for carrying out the several purposes of the present technology. It is, therefore, that the claims be regarded as including such equivalent constructions insofar as they do not depart from the spirit and scope of the present technology.

It is therefore an object of the present technology to provide a new and novel protein nanoparticles to increase exercise endurance, functionality and longevity in cancer and non-cancer patients that has all of the advantages of known stem cell mobilization and none of the disadvantages.

It is another object of the present technology to provide a new and novel protein nanoparticles to increase exercise endurance, functionality and longevity in cancer and non-cancer patients that may be easily and efficiently manufactured and marketed.

An even further object of the present technology is to provide a new and novel protein nanoparticles to increase exercise endurance, functionality and longevity in cancer and non-cancer patients that has a low cost of manufacture with regard to both materials and labor, and which accordingly is then susceptible of low prices of sale to the consuming public, thereby making such protein nanoparticles to increase exercise endurance, functionality and longevity in cancer and non-cancer patients economically available to the buying public.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is a graphical view of the effect of treatment to cytokine IL-17A level.

FIG. 2 is a graphical representation of the effect of treatment to Fractalkine level.

FIG. 3 is a graphical representation of the effect of treatment to MCP-1 levels.

FIG. 4 is a graphical representation of the effect of treatment to RANTES level.

FIG. 5 is a reproduction of FIG. 1 of the graphical representation of CD34+ cell ratio (%) in total mononuclear cell population (n=4/group) as illustrated in the poster presentation comparison at the ORS 2024 Annual Meeting.

FIG. 6 is a diagram of the oxidation of glucose using NAD (P) as co-factor.

### DETAILED DESCRIPTION OF THE EMBODIMENTS

While the above-described devices fulfill their respective, particular objectives and requirements, the aforementioned devices or systems do not describe a protein nanoparticles to increase exercise endurance, functionality and longevity in cancer and non-cancer patients that allows enriching stem cells and pluripotent cells by intravenously administration of protein nanoparticles.

A need exists for a new and novel protein nanoparticles to increase exercise endurance, functionality and longevity in cancer and non-cancer patients that can be used for enriching stem cells and pluripotent cells by intravenously administration of protein nanoparticles. In this regard, the present technology substantially fulfills this need. In this respect, the protein nanoparticles to increase exercise endurance, functionality and longevity in cancer and non-cancer patients according to the present technology substantially departs from the conventional concepts and designs of the prior art, and in doing so provides an apparatus primarily developed for the purpose of enriching stem cells and pluripotent cells by intravenously administration of protein nanoparticles.

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

A goal of the present technology is the increase in the concentration of useful pluripotent cells in a patient (after the intravenous administration of protein nanoparticles) particularly in the voluntary muscle system, so that the muscle mass can be built up resulting in improved exercise endurance, in the patient's ability to live a productive life during “old age” and in prolonging life span.

Regarding protein nanoparticles or nanospheres, Yen has disclosed their manufacturing process and numerous applications: e.g. “Albumin nanoparticles to augment stem cell function in vivo” U.S. Pat. No. 11,260,109, issued Mar. 1, 2022; “Nanoparticles for the therapeutic treatment of radiation-induced skin ulcers” U.S. Pat. No. 11,260,110, issued also on Mar. 1, 2022; “Protein Nanospheres to treat harm from multiple trauma” US Patent Publication No. 20230241182, published on Aug. 3, 2023. The name of the biological product is called Fibrinoplate-S(FPS) which is a ready-to-use suspension containing the active substance which is called Fibrinogen-coated Albumin Spheres (FAS).

Yen disclosed that certain protein nanoparticles or nanospheres can be administered via the intravenous route, which will result in the attachment of such nanoparticles to the cells residing in various locations in the body. For example, nanospheres have been observed to attach to the endothelium of blood vessels. In addition, recent experiments showed that these nanospheres (called “Fibrinogen-coated albumin spheres, FAS”) can also attach to bone marrow cells inside the bone marrow. We expect that these nanosphere may attach to certain cell populations in the blood, e.g. stem cells that are circulating in the peripheral blood. Furthermore, since stem cells are found in many other tissues, including the muscles and the adipose tissues, we expect these protein nanosphere may also be able to attach to these pluripotent cells already residing in mature tissues of the body.

The attachment of protein nanoparticles such as FAS can have profound effects on the attached cells. It has been observed that the administration of FAS intravenously can increase the concentration of CD34+ (stem) cells inside the bone marrow, followed by increased concentration of certain “more mature” cells (e.g. monocytes) in the peripheral blood. Some of these observations have been reported in a non-provisional patent application titled “Protein Nanospheres to Treat Harm from Multiple Trauma.”

Several separate experiments using different animal models suggest that the intravenous administration of FPS can lead to the mobilization of stem cells whether these stem cells originate from the bone marrow or from endothelial cells or from other locations. In the prior arts and Yen disclosures, the aim was mainly to use the protein nanoparticles to mobilize stem cells to heal wounds. The data showed that FAS can mobilize stem cells which would later migrate to the wound site and then these stem cells would differentiate into the mature tissues (e.g. muscles, blood vessels, skin, nerves) to bring about the healing of the entire wound site. In addition to the macroscopic observation that the FAS-treated group had accelerated healing of the wounds compared to the control group, CD34+ stem cells could still be detected in the biopsies of the wound (or healed-wound-area) and these CD34+ cells are in far greater abundance in the FAS-group than in the saline-group. This is particularly interesting because if the wound had healed, those stem cells that brought about the healing are supposed to have differentiated into mature cells. During differentiation, the stem cells that have arrived at the wound should all have shed their original CD34+ cell marker (because the differentiated and functional cells typically will have other cell markers, but not the CD34 marker.) The fact that CD34+ cells are still observed in the healing (or healed) wound biopsy, far more in the FPS-group than in the saline-group, and at many weeks after one dose of FPS administration, suggests strongly that there must be an overabundance of CD34+ stem cells having arrived at the wound at the various stages of healing, many of which still retain their “primitive” CD34+ cell marker, even at the time of biopsy (e.g. on day 51 after FPS administration) when the wound is observed macroscopically to have almost completely healed.

However, it should be noted that Yen had not discussed the effect of the administration of FAS in healthy subjects with no wounds or with no dysfunction, whether the subject is young or in old age. The disclosure in this patent application is to reveal the effect of the intravenous administration of protein nanoparticles on the healthy body, in particular, the effect on MDSC and other muscle-related pluripotent cells, which will result in stronger and more capable muscles (able to endure for longer or more vigorous exercises). This will be beneficial to the patient regardless of age, for their daily living and for heavy labor. An additional benefit is the reduction of morbidity such as falling or bone fracture due to weak muscles. Another benefit is the increased longevity due to, or in addition to, productive life.

Specifically, regarding the mobilization of stem cells by the administration FPS in healthy animals, a dose of FPS administered intravenously has been shown in separate experiments to result in a chain of events that are linked to each other timewise (in a sequential manner.) (a) The data showed that the active drug substance (FAS) can enter from the vascular compartment into the bone marrow compartment of the bone marrow of healthy mice. This experiment used FITC (fluorescein isothiocyanate)-labeled FAS, which was administered intravenously into healthy mice. Following isolation of bone marrow cells from these mice, FITC-FAS can be seen in flow cytometer to be still attached to the isolated bone marrow cells. (b) In separate experiments, unlabeled (original) FAS administered intravenously to mice has been shown to result in an increase of CD34+ cells in the bone marrow. (c) Again, in separate experiments using mini-pigs, an increased concentration of CD34+ cells can also be detected in the blood soon after administration of FPS. (d) In rats suffering from skin injury induced by a high dose (25 Gy) of local irradiation onto the skin causing a visible wound within a month, two doses of FPS given after on day 1 and day 2 after the appearance of the skin lesion will result in the obvious healing of the wound within 9 days and an abundance of CD34+ cells can still be detected in the biopsy obtained at this late stage. (e) In minipigs irradiated at the skin with 50 Gy of irradiation, around 50 days after the administration of FPS, the skin biopsy of the FPS-group showed recovery of blood vessels. It is well known that high doses of irradiation can greatly damage blood supply by the destruction of blood vessels. In some areas in the biopsy, the FPS-group showed (by histological staining) normal intensity and distribution of CD31+ cells (which labels endothelial cells): showing that neo-vascularization is essentially complete by that time, giving the skin of the FPS-group a chance to receive normal nutrients from the blood (because the blood vessels are now functional in that area due to the healing effects of the FPS, via the mobilization of stem cells.) However, these previous disclosures do not include or discuss the effect of FPS or FAS on the muscle mass or the muscle-related stem cells such as MDSCs or SCs.

The beneficial effects of FPS can also be anticipated from its effect on cytokines, specifically the suppression of pro-inflammatory molecules induced by a wound (including radiation-induced wounds) or stress, and FPS's promotion of anti-inflammatory cytokines. In a report to the NIH (Report to NIAID (SBIR GRANT NUMBER 1R43A1142964-01, “A multivalent, easy-to-use product to mitigate and treat radiation exposure” PI: Yen, Richard, 2019) the data showed the positive effect of FPS on the cytokine in rats injured by irradiation. We expect a similar effect on the healthy patient under stress (even those which have no visible wound.) This is because stress, including vigorous muscle exercise can induce the production of inflammatory cytokines.

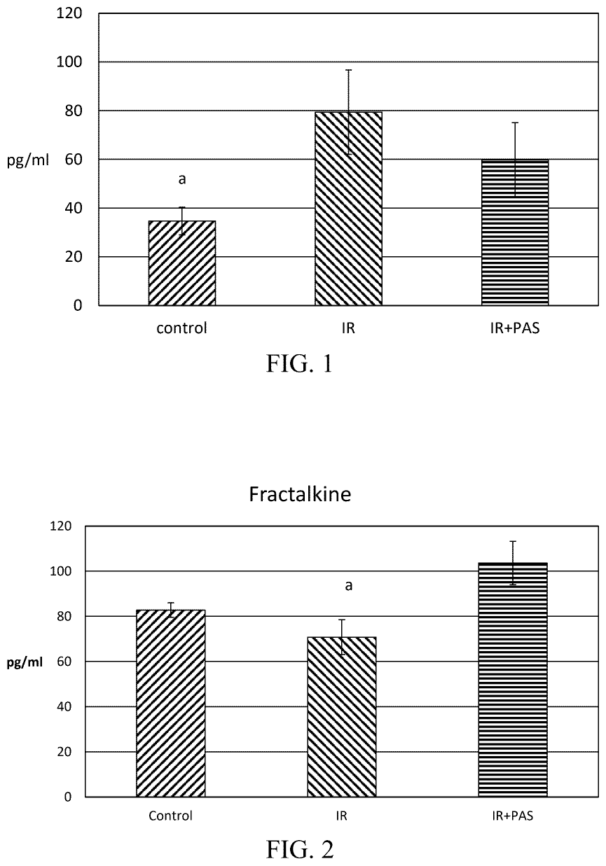

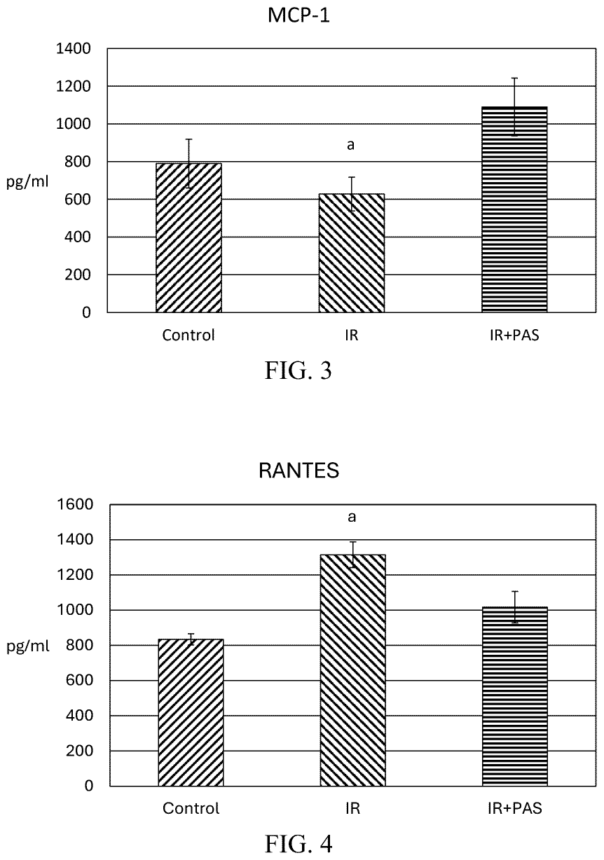

A detail analysis of the cytokines was performed as follows: samples of frozen serum collected from the rats were thawed and evaluated using the Multiplex immunoassay. While we attempted to measure all 22 cytokines in all rats, several of the cytokines were not quantifiable or no significant difference between the saline-group vs the FPS-group. However, several cytokines were significantly altered by FAS treatment. In this experiment, “control” means “control animals with no irradiation”; “IR” means “irradiated animals treated with saline”; “IR+FAS” means “irradiated animals treated with FPS”. FIGS. 1 to 4 showed the effect of treatment on various cytokines (the bar being the standard deviation, “a” means the mean of the group is significantly different from the mean of the other groups.) The data showed that 25Gy of irradiation would cause the elevation of IL-17 A, a proinflammatory cytokine, in the IR group compared to control, and FAS reduced its expression level in the blood in the IR+FAS group (FIG. 1). Another cytokine called Chemokine fractalkine (FKN), which plays an important role in stem cell development was significantly depressed in the IR group, but increased in IR+FAS group. For a third cytokine: the expression of monocyte chemoattractant protein-1 (MCP-1)—it was shown to be down regulated in the irradiated (IR) group but enhanced in IR+FAS treated group which showed increased modulation of immune response, which would be beneficial to the animal. Finally, FIG. 4 shows the effect of treatment on RANTES, which are pro-inflammatory cytokine known to be secreted by endothelial cells. Our study showed a significant increase of RANTES expression in the irradiated (IR) group but FAS ameliorates the animal's inflammatory response in the IR+FAS group. In short, FPS treatment collectively appears to increase anti-inflammatory responses and suppress pro-inflammatory responses of the body.

The administration of FPS has a positive effect on the healing of “hard tissues” such as the repair of severe bone fracture (whether the patient is young or old.) Recently, the intravenous administration of FAS has also been shown to accelerate the mineralization of healing bones in vivo after a fracture. At the same time, it was discovered that there are more than twice the number of CD34+ stem cells at the callus site of the FAS-treated group compared to the saline-control group. This project was funded by NIH grant: 1R43AG072919-01A1. Therefore, FAS appears to be effective in the healing of soft-tissue wounds such as a skin wound, as well as hard tissue injuries such as a bone fracture. The mechanism of healing in all of the above cases is most likely due to the mobilization of CD34 cells, some of which became blood vessels with healthy endothelium (staining positive for CD31 cell marker) and others became mature cells such as nerves and muscles, seen in the slides of the biopsies by standard methods of staining. We do not know yet the extent cytokines exert in wound healing: one can predict that a decrease in pro-inflammatory response would be helpful in the body's ability to heal. FPS tends to be beneficial in both the mobilization of stem cells to the wound as well as concurrently the suppression of pro-inflammatory cytokines.

All of the above data showed that (a) FAS administered intravenously can accelerate the healing of a variety of wounds; (b) the healing or healed wound would still show an abundance of CD34+ stem cells, despite the fact that the visible healing of the wound (by macroscopic observation as well as histological studies of the biopsies) would require differentiation of the (originally) stem cells which had arrived at the wound. During the maturation of these stem cells, they should have lost the cell marker CD34; but there are still enough CD34+ cells leftover at the wound site which have not shed their CD34 cell markers: much more so in the FPS-treated group than the control saline-treated group. The data suggest strongly that there must be an abundance of CD34+ cells having arrived and stayed at the wound site, during the entire process of healing; from the early phase to the late phase of healing.

The following is an abstract which disclosed the effect of FPS on the healing of a “hard-wound” which is a surgically induced bone fracture in old rats. The abstract had been sent to the Orthopedic Research Society, which indicated that it had been accepted for publication. The abstract has been chosen to be presented as a poster presentation at the ORS 2024 Annual Meeting in Long Beach, California, February 2-6. Details regarding the presentation are noted below:
POSTER SESSION #: PS1-008
POSTER CATEGORY: Bone-Injury and Healing
POSTER #: 472
POSTER TITLE: Fibrinogen-coated Albumin Spheres To Accelerate Bone Healing In Older Rats
AUTHORS: Sang-Hyun Park; Istvan Kovanecz; Edward Ebramzadeh; Richard Yen
The content of the abstract is as follows:
   Fibrinogen-coated albumin spheres to accelerate bone healing in older rats
Sang-Hyun Park, Ph.D.1, Istvan Kovanecz, Ph.D.2,3, Edward Ebramzadeh, Ph.D.1, Richard Yen, M. D.4
   1 The JVL Sr. Orthopaedic Research Center, Orthopaedic Institute for Children, Dept. of Orthopaedic Surgery, UCLA, Los Angeles, CA,
   2Dept. of Surgery, The Lundquist Institute, Torrance, CA, 3Dept. of Urology, UCLA, Los Angeles, CA, 4Fibroplate, Inc. Las Vegas, NV.
   DISCLOSURES: Study was supported by NIH 1R43AG072919-01A1. S-H. Park (N); I. Kovanecz (N); E. Ebramzadeh (N); R. Yen (N)

### INTRODUCTION

Fracture healing results from a complex, sequential cascade of cellular events. Although the exact regulatory mechanisms for fracture healing are not yet fully understood, neo-vascularity as well as osteoblast recruitment at the callus site appear to play key roles in successful healing. We investigated a novel intravenous injectable biological product based on Fibrinogen-coated Albumin nano-Spheres (FAS). Extensive preliminary studies demonstrated multi-valent properties of FAS, including the ability to support and accelerate wound healing of multiple ulcerated-necrotic tissues, by promoting the mobilization of different lineages of progenitor cells, particularly Endothelial Progenitor Cells (EPCs), and decreasing levels of pro-inflammatory cytokines (including IL-6) in the blood. One of the main factors that affect bone healing in the elderly is the decline in the number and function of skeletal stem cells, which are essential for bone regeneration. We hypothesized that FAS treatment could improve the survival and differentiation of CD34+epithelial stem cells in the bone marrow, as well as their ability to enhance fracture healing. Therefore, the goal of the present study was to evaluate the efficacy of intravenous FAS administration on fracture healing in a small animal model.

### Methods:

Unilateral mid diaphyseal femoral fractures were created in 56 male rats (15-month old male Fischer 344) and fixed with an intramedullary nail, approved by the Institutional Animal Care and Use Committee. Rats were treated either with a saline control (2 ml/kg) or Fibrinogen-coated albumin spheres (2 ml/kg=16 mg spheres/kg), both IV-administered on days 0, 3, 6 and 9 post-fracture. Healing was assessed on days 21 and 35 post-fracture. The newly formed mineralized callus (>500 mg/cm³) properties (volume and mineral content) of 3.6 mm long bone segment at the fracture line were quantified by qCT at days 21 and 35 (n=10/sub group). At day 35, the biomechanical properties of the fracture sites, including maximum torque and torsional stiffness were evaluated. Paraffin-embedded, decalcified femur specimens containing the fracture (n=4/subgroup) site were sectioned and stained with CD34 rabbit polyclonal antibodies, peroxidase-conjugated mouse anti-rabbit IgG, DAB substrate, and hematoxylin counterstain. The percentage of CD34+ cells in the mononuclear cell population was counted at 100× magnification, six fields per section. Data were analyzed for treatment efficacy with one-way ANOVA and paired t-test.

### Results:

Overall, the study group demonstrated faster callus development than control group at the fracture site. At 21 days, the FAS study group was approximately 30% higher in mineral content and mineralized callus volume (p=0.01) at the fracture site than the control group. (Table 1). However, by 35 days, the callus development was comparable for both groups. Specifically, there were no differences in mineral content or mineralized callus volume at the fracture site by day 35. At day 21, neither the control group, nor the study group exhibited measurable stiffness at the fracture site under biomechanical evaluation. However, by day 35, 5 of the 9 control group specimens and 4 of the 9 study group specimens exhibited measurable torsional stiffness and strength under torsional testing. The mean values for maximum torque was higher in study group than control group (164.3 vs 209.7 Nmm, p-0.048); however torsional stiffness was higher in control group than study group (5.89 vs 3.23 Nmm/deg., p=0.023). There was a significant increase in the percentage of CD34+ cells among total mononuclear cells in the 21 day study group compared to the control (4.13±0.28 vs.1.51±0.15, p<0.0001), as well as in the 35 day groups (4.45±0.43 vs. 2.19±0.2, p<0.0001), as best illustrated in FIG. 5.

### DISCUSSION

Our results confirmed the previous findings that intravenous FAS administration induces CD34+ stem cell activation and migration via circulation to the injury site during the entire fracture healing period. Further, FAS administration boosted mineralized callus development at the fracture site during the early fracture healing period. However, early administration of FAS did not appear to significantly increase mineralized bridging at the callus site or torsional strength or stiffness at 35 days. This finding was likely affected by the timing and dosage of the treatment drug. While the preliminary data is promising, future studies are needed to evaluate the efficacy of FAS on fracture healing.

### Significance/Clinincal Relevance:

Our results confirmed the previous findings that intravenous FAS administration induces CD34+ stem cell activation and migration via circulation to the injury site during entire fracture healing period and early callus development.

TABLE 1
Newly formed hard callus at the osteotomy site (3.6
mm long segment) at Day 21 and 35 (mean ± S.D).

| | Newly Formed Hard Callus only (>500 mg/cm³) | | | |
|---|---|---|---|---|
| | Callus Volume (mm3) | | Mineral Content (mg) | |
| Group | Day 21 | Day 35 | Day 21 | Day 35 |
| Control | 11.69 ± 2.01 | 16.37 ± 2.62 | 7.50 ± 1.38 | 10.70 ± 1.91 |
| FAS Group | 15.50 ± 4.10 | 16.29 ± 6.30 | 9.79 ± 2.57 | 10.62 ± 4.10 |
| P value | 0.011 | 0.486 | 0.015 | 0.479 |

n = 10 for each subgroup (except 21 day control group, n = 9)

Reference: 1. Mao et al. Radioprotective efficacy of fibrinogen-coated albumin spheres (FAS) against radiation-induced skin damage. 65th Radiation Research Society Annual Meeting, 2019.

The disclosure in the above abstract discussed the effect of FPS on bone or bone-healing. It did not discuss the effect on the muscles, which is the object of this patent application.

Regarding the method to identify MDSC or other muscle-related pluripotent cells, a recent publication (Tang X, Su X, Zhong Z, et al. “Molecular mechanisms involved in TGF-β1-induced Muscle-derived stem cells differentiation to smooth muscle cells [in English].” Am J Transl Res. 2019; 11 (8): 5150-61) showed that SCA-1, CD34, and CD45 may be reasonable markers for the identification of MDSCs. It is generally established that MDSCs express the mesenchymal stem cell markers CD73, CD90, and CD105 [22], and some other positive markers have been revealed, including CD29, CD44, and CD133 (Liu et al.)

Since stem cells or other pluripotent cells residing in the muscles can also carry the CD34+ cell marker on their surface, the CD34+ cells in the skin wound or bone callus described above may have come from (a) the bone marrow, or (b) from muscle-derived stem cells which are already residing in the muscle at the time of activation, or a combination of (a) and (b). We expect that in non-wounded animals, FPS can have effects on both bone marrow CD34+ cells and/or muscle-residing CD34+ cells.

In this disclosure, when we reveal the positive effect of FAS on the host. However, we do not rule out the possibility that the benefit comes from the supportive cells in the bone marrow or the supportive cells located in the other “stem cell sites” (including the endothelium or other perivascular sites or in the muscle or adipose sites). In addition, the benefit can come from cytokines or soluble factors generated by any of the cells. However, in most cases, we expect the benefit of FAS administration to come from the direct effects of FAS on the stem cells (regardless of their sites of origin) to cause them to regenerate additional stem cells or to divert into the differentiation pathways, leading to mature cell types in their final destination, which result in the desired effect in the patient.

With respect to cancer patients, whether the patient has hematopoietic cancers or non-hematopoietic cancers, we expect FAS to exert benefits to these patients without the side effects of (a) promoting cancer growth, (b) inhibition of any cancer treatment effectiveness (whether by radiation or chemotherapy or any other cancer treatment method.)

It has been published in open source that many cancer patients have impairments before their cancer treatment which negatively impacted their cancer treatment, e.g. by hematopoietic stem cell transplantation. In a paper published in Transplant Cell Ther 2022 May 17;28 (8): 498, titled “Geriatric Assessment Reveals Actionable Impairments in Hematopoietic Stem Cell Transplantation Candidates Age 18 to 80 Years” the authors (Meagan V Lew et al.) made the following statements in their Abstract:
“Allogeneic hematopoietic stem cell transplantation (HCT) is a potentially curative treatment for both malignant and nonmalignant hematologic diseases; however, reported rates of treatment-related mortality approach 30%. Outcomes are worse in patients who begin HCT with functional impairments. To detect such impairments, a geriatric assessment (GA) is recommended in adults age ≥65 years. Younger HCT candidates also may be impaired because of chemotherapy regimens pre-HCT. Therefore, we hypothesized that GA can be beneficial for adult patients of all ages and subsequently created a clinical pretransplantation optimization program to assess all HCT candidates using a modified GA. One-hundred fifty-seven patients were evaluated in 4 functional domains—physical, cognitive, nutritional, and psychological—at 2 time points prior to HCT—new patient evaluation (NPE) and sign-off (SO)—between October 2017 and January 2020. At NPE, 80.9% of the patients had at least 1 domain with a functional impairment, and physical (P=.006), cognitive (P=.04), and psychological (P=.04) impairments were associated with an increased likelihood of not proceeding to HCT. In addition, patients age 18 to 39 years were more likely than older patients to have a physical function impairment (P=.001). Between NPE and SO, 51.9% of the patients had resolution of 1 or more impairments, and nutritional impairment at SO was predictive of worse overall survival (P=.01). Our study shows that GA can identify functional impairments in patients of all ages. Early identification of impairments could facilitate referrals to supportive care and resolution of impairments prior to HCT, suggesting that GA could be recommended for HCT candidates of all ages.”

Note: It is very impressive that with only “referrals to supportive care” some 51.9% of the patients had resolution of 1 or more impairments, thus improving their chance of advancing to HCT treatment and possibly better outcomes after HCT treatment (which was not studied in this NIH grant.) We expect that by the augmentation of benefits using a physical entity (a “medication”) which is protein nanoparticles, particularly FAS, the improvements will be obvious in any of the four categories studied, or additional parameters, so that more patients can receive or are willing to receive HCT; and that they will have better outcomes from HCT. Even though we mention HCT in this disclosure as an example, we expect improvements in other modalities of cancer treatments by the administration of FAS. The improvements will be seen with or without referral to supportive care or that the patient actually completed the exercises or remedies recommended by these “supportive care” providers.

### Experiments

EXPERIMENT ONE: Effect of FPS administration on muscle mass and exercise endurance in healthy non-injured mice

Purpose of this Pilot Study

The hypothesis: the intravenous administration of FPS into old mice will increase their muscle mass leading to increased exercise endurance (and secondarily, to longevity.) The same scheme of FPS administration is beneficial to young healthy individuals.

### Design of the Study

20 old mice (68 to 70 weeks old, C57BL/6J) will be divided into 2 groups (10 each, 5 male, 5 female). Each will be identified and the results are compared (a) within the same animal, before vs after treatment; and also (b) the value of the whole saline-group compared with the whole FPS-group at various time points. Group I will be administered FPS; Group II, saline.

There are two kinds of exercises: (a) Strenuous Exercise: exercise until animal fails the test, e.g. cannot exercise or falls off the exercise machine. (b) Mild Exercise, which is routine exercise intended to maintain healthy muscles.

Every mouse will be subjected on day-3 to one Strenuous Exercise, to obtain baseline value of how much each mouse can exercise.

On Day 0, each mouse will be subjected to Strenuous Exercise—This time it is intended to produce deep stress; then to Group I the FPS is administered (2 mL per kg) at 1 to 2 hour post-Strenuous Exercise. To Group 2, saline is administered (2 mL per kg) at also 1 to 2 hour post-Strenuous Exercise. The mice are then allowed to rest or do whatever they want freely.

Subsequently, all mice will be administered intravenously weekly doses (2 ml per kg each: i.e. 2 ml of FPS (equal to 16 mg spheres) per kg; or 2 ml of saline per kg: in short, the administration of injections will be done on Day 0, 7, 14, 21.

Mild Exercise every day to keep healthy except on days of Strenuous Exercise.

### Strenuous Exercise on Day 21 and Day 42.

If there is an observable difference between Group I and Group II in terms of muscle mass on the legs or exercise tolerance on Day 42, keep 6 mice (6 each group) alive till much later or their natural death to evaluate natural longevity. Do histology studies on muscles.

If there is no major difference between Group I and Group II by Day 42, sacrifice all mice and do histology study: in case there is improvement in the muscle groups but the improvement is not reflected in exercise tolerance or endurance.

Days of Strenuous Exercise and treatment (Table 2):

TABLE 5

| | Day | Day | Day | Day | | End of |
|---|---|---|---|---|---|---|
| | 0 | 7 | 14 | 21 | Day 42 | life |
| Exercise | yes | yes | yes | yes | yes | One |
| till mice | | | | | | more |
| fail test | | | | | | exercise |
| | | | | | | endurance |
| | | | | | | test near |
| | | | | | | end of |
| | | | | | | life |
| Admin | yes | yes | yes | yes | No drugs | Option: |
| saline vs | | | | | or saline, | Keep 6 |
| FPS 2 hr | | | | | sacrifice | mice each |
| after test | | | | | for muscle | group (3 |
| on same | | | | | biopsy | male, 3 |
| day | | | | | study if | female) |
| | | | | | there is a | till end |
| | | | | | difference | of life? |
| | | | | | in endurance | |

### Data to Collect

Duration of exercise tolerance during Strenuous Exercise.

Regardless of whether there is a difference between FPS-group vs saline at Day 21 or Day 42, measure muscle mass and muscle stem cells (MDSC) and satellite cells (SC) concentration in biopsy (plus any other meaningful parameters.)

### Results

Preliminary results show that weekly injections (intravenously) of FPS as described resulted in improved exercise tolerance in old mice in Group I, when compared to Group II. Comparison of Strenuous Exercise duration of each mouse before vs after FPS (or saline) treatment showed improvement (paired evaluation statistically) significantly. However, there is much variation within the groups and so Group I vs Group II showed an improvement of Group I over Group II, but may not be statistically significant. An increased number of animals (N>10 per group) may show significance in statistics between groups. We are waiting for results of muscle biopsy, staining of MDSC and SC in the muscles. We expect an improvement in muscle mass and the number of MDSC and SC in the FPS group compared to the saline group. We also expect the same conclusions in the young animal population as found in the old animal population.

Any study in “Longevity” in mice will need a thorough forensic study of the carcass. This is because longer life may be due to factors related to suppression of diseases, while mice living shorter lives are due to diseases or other old-age related (but not directly longevity-related) factors which are the causes of demise for those which showed “shorter life span.” Therefore, the verification of “true longevity” will take a long time and a lot of effort. Preliminary data, however, show that the FPS-group is showing longer life span than the saline-group.

The data suggest that the same results may be found in the human population. However, it is not practical to obtain muscle biopsies in the human populations, especially in the frail and old population. We will depend on exercise tolerance for near-term assessment of the effect of FPS administration in the older human population.

In terms of commercialization, we intend to promote the use of FPS in the pet market first. This is because many pet-owners will value the possibility of extended (and healthy) life for their pets. Data on the effect of FPS to prolong life in animals are easier to obtain than in human populations.

In the younger population, a study of prolonging life will be difficult because of the time involved. We may aim at the body-building population where the client is primarily interested in observable increase in body mass; and the weight-lifting population where the amount of work can be objectively measured by the actual increase in weight-lifting ability.

EXPERIMENT TWO: Effect of FPS administration on the Functional Observational Battery Tests conducted in older mice.

### Purpose:

To demonstrate the benefits of FPS administration on the functional (behavior, psychological) aspects of older individuals in addition to increase in exercise tolerance or muscles mass.

### Methods:

Administration of FPS or saline was conducted in a scheme similar to Experiment One.

The following Table 3 lists a battery of test for functionality which can be observed.

TABLE 3
Home Cage Observations^{a}
(Posture/activity/palpebral closure/abnormalities
Males
Females
Ease of Handling^{a}
(During removal from cage/when held)
Males
Females
General Appearance^{a}
(Lacrimation/salivation/coat appearance)
Males
Females
Open Field Activity^{a, b}
(Latency/total number squares entered/total number extra lines crossed/
freezing/freeze time/sniffing/grooming/headsearch/supported rear/
unsupported rears/arousal/gait description/number fecal boluses and urine
spots/diarrhea)
Males
Female
Reflexes^{a}
(Acoustic/approach/touch/tail pinch/righting reflex/proprioceptive
positioning/forelimb & hindlimb extension/sensory flexor reflex/muscle
strength flexor reflex/reaction to pain flexor reflex)
Males
Females
Physical Measures^{b}
(Grip strength/landing foot splay/body temperature/body weight)
Males
Females
^{a}Kruskal -Wallis followed by Wilcoxon
^{b}Analysis of Variance followed by Duncan's procedure

### Results:

Preliminary data suggested that the FPS-treated group performs better than the saline-treated group in many of the items tested. We expect similar results in the human population. However, in the human population we can do additional tests to show that FPS can improve the psychological profile of the individual because the FPS-treated persons would feel better due to their healthier status and better social interactions, in addition to any improvements in their cognitive abilities.

### Conclusion:

Weekly doses of FPS administered in animals can improve their functional capacity, and possibly cognitive performances.

EXPERIMENT THREE: Effect of adding cofactors orally on the beneficial effects of FPS administration in young and old adults.

### Purpose:

To evaluate if there is any synergistic effect with FPS when the individual also takes supplements containing co-factors that are known to increase cellular energy levels.

### INTRODUCTION

For the body to function, there must exist the proper cell/tissue/organ structure and their ability to function. To do all these, there must be energy to build/repair the structures and enough energy for the structures to exert their designed functions. Fundamentally, all the energy needed for these functions comes from the food we eat. The food is broken down by the digestive system and then converted mainly into glucose, which can be converted inside the cell (at the mitochondrial level) to the cellular storage molecule called ATP (Adenoside Tri-Phosphate).

All these biochemical reactions are complicated. However, in almost all steps, cofactors are needed for the reaction to be completed successfully, as explained below by the example of glucose dehydrogenase.

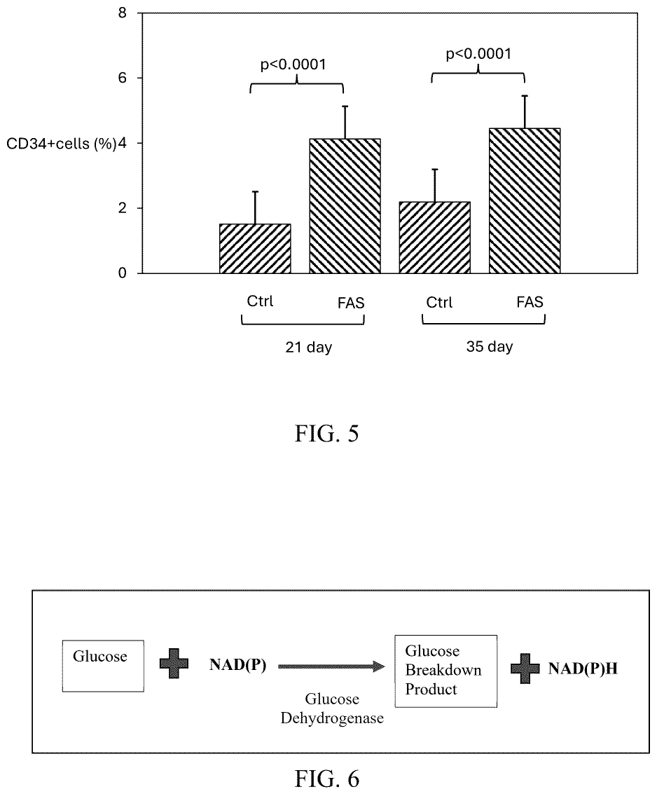

As shown in FIG. 6, oxidation of any molecule can be understood as: (a) addition of an oxygen atom to the molecule, or (b) removal of a hydrogen atom from the molecule. For biochemical reactions, most of the oxidative steps are done by removal of hydrogen molecules, which will facility the breakdown of the original molecule, such as glucose. The enzyme Glucose Dehydrogenase is called a “dehydrogenase” mainly because it removes hydrogen from substrate molecules—in this case, glucose molecules. In order for the reaction to occur, “someone” has to “take the hydrogen atom.” In this case, the “receiving molecule for the hydrogen” is Nicotinamide Adenine Dinucleotide (NAD). An alternative form for NAD is NADP (Nicotinamide Adenine Dinucleotide Phosphate). In FIG. 6, the co-factor is presented as NAD (P) to mean either NAD or NADP will work, because they both perform as “hydrogen sinks” (keepers of hydrogen molecules.)

The enzyme Glucose Dehydrogenase exists in the body mainly to facilitate the reaction going from left to right, mainly because the concentration of Glucose is high in the body (especially after a meal.) The result of this reaction is the formation of the “glucose breakdown product.” We will not go into the details of these reactions, because the main point is not the breakdown products of glucose, but the formation of NADH as a result of the participation of NAD; or the formation of NADPH as a result of the presence of NADP.

It can be readily seen by people knowledgeable in biochemistry that several conditions can increase the rate of the reaction (using glucose as an example.) The conditions are: (a) an increased concentration of the enzyme, (b) an increase in the concentration of the “substrate.” In this case, glucose is considered the substrate, because that is what scientists are interested in studying (as to what will become of this glucose molecule after it is broken down.) Other conditions (c) would include the pH, presence of other salts, or a slight increase in temperature. However, another important condition is often ignored (d)-that is the concentration of the “co-factor.” In fact, NAD and NADP are called “co-factors” (or hydrogen sink, not as bad as named “kitchen sink”) because they are present in most cellular environments, and not specific to any particular reaction. However, when these “co-factors” are in low concentrations—and they will be depleted soon by the activity of the glucose dehydrogenase in the presence of a high concentration of glucose—the reaction will not proceed (regardless of how much more glucose or the enzyme is added to the reaction mixture.) Therefore, these co-factors should be considered as very important in the continuous function of all the enzyme systems. Experimental data have shown that an abundance of NAD (higher than normally used) will improve the rate of the enzymatic reaction even when “standard concentrations” of glucose and Glucose Dehydrogenase are used.

The generation of co-factors in the body and in the cells of the body involves very complicated reactions. However, if we focus on the most important ones, such as NAD or NADP, we can see that an increased supply of the precursors to these co-factors will help all the cellular reactions to go up faster or go on longer. This is because an abundance of the “precursors” will lead to an abundance of “co-factors” which will allow faster and more efficient increase in cellular metabolism, which will provide more energy to build more muscles mass or its function as expressed in exercise tolerance. There are two important precursor molecules to the formation of NAD: (a) Nicotinamide Riboside (NR); (b) Nicotinamide MonoNucleotide (NMN). It has been found that oral intake of up to 1000 mg of NMN is quite safe to a normal sized human being. In this disclose the term “co-factor precursor” can mean any of the molecules that can increase the concentration of NAD or NADP in the body or in the cells of the body.

We intend to study the effect of an increased level of energy supply (by providing an increased concentration of the precursors to the cellular co-factors) on the ability of protein nanospheres to mobilize stem cells from the bone marrow (or other sites) in normal healthy individuals leading toward benefits to the muscles, to increase muscle mass, exercise tolerance and longevity.

In general, co-factor precursors are sold today as nutritional supplements or health supplements. There is practically no study on their effect on stem cells or their effects on the safety or effectiveness of protein nanospheres on the body. Therefore, the combined use of co-factor precursors (orally) with FPS (intravenously) is novel and non-obvious.

### Materials and Methods:

The protocol of Experiment One was used here, except that mice were fed orally with their standard food, on a daily basis, 20 mg of NR per kg. The Strenuous Exercise and routine exercise routines were conducted in the same way as in Experiment One. There are 4 groups (each 10 mice): Group A: FPS alone; Group B: NR alone; Group C: FPS plus NR; Group D: saline alone (no FPS, no NR).

### Results:

Preliminary results show that Group D is the baseline. Group B is no better than Group D. Group A is better than Group B and Group D. Remarkably, Group C is the best, better than even Group A: the data suggested that there is a synergetic effect when co-factor precursors are consumed by the individual in conjunction with FPS administration.

### Comments:

We used NR here as an example of co-factor precursor. Other co-factor precursors such as NMN are expected to be similarly useful. Again, the effect can be seen on both young and old individuals. The data on longevity are pending. But we expect Group C to be better than Group A, which is better than Group B and D.

EXPERIMENT FOUR: Effect of FPS administration on the muscle mass and exercise in conditions of low gravity and space irradiation.

### INTRODUCTION

Main concerns for long-term deep manned space missions include health-risks associated with simultaneous prolonged exposure to microgravity environment and to cosmic radiation. Altered gravitational environment represents a unique challenge for biological systems that have evolved against a constant and substantial gravitational background. It is well known that exposure to actual and/or simulated microgravity causes various physiological adaptations in the life organisms. Astronauts are known to have difficulties walking on earth immediately after a prolong stay in space, because of their lack of exercise and because of the microgravity environment.

### Purpose:

This experiment intends to study critical questions that the NASA Space Biology Program may be interested in. Preliminary studies in mice have shown that environmental conditions similar to space travel will increase oxidative stress and induce adverse micro-vessel remodeling and immune response. Fibrinogen-coated albumin spheres (FAS) is a novel medical product known to be able to mitigate these known problems. It is also capable of interacting in vivo with the endothelium of blood vessels and to mobilize stem cells from the bone marrow. We intend to study here the effect of FAS administration on the body's muscle mass under conditions of space flight, as reflected from exercise tolerance and any biomarker related to stem cell mobilization to increase muscle mass.

Our hypothesis is that FAS administered intravenously will mitigate spaceflight and simulated (i.e. a condition created on earth which mimics space flight conditions) microgravity/space radiation-induced damage to the muscles. A second hypothesis is that FAS can accelerate tissue repair by activating endothelial/or bone marrow stem cells and maintaining muscular and vascular integrity.

Specific goal 1: Evaluate physiological and pathological changes in organ/tissues (specially muscles) of mice after exposure to simulated spaceflight environment on earth (and after live animals return from space, if possible) with and without FPS treatment.

Specific goal 2: Determine physiological and pathological changes in organ/tissues of mice after exposure to dose of cosmic/space radiation and simulated microgravity using hindlimb unloading (HLU), with and without FPS treatment.

Specific goal 3: Integrate pathway-based evaluation of molecular profiling with genomics and metabolomics approaches for risk assessment to compare tissue response following space flight condition and treatment.

### Materials and Methods:

For ground-based study, mature female mice will be used in this study. Due to the difficulties of repeated drug administration in space, the animals will be administered FAS or saline only once. The mice will be treated with an infusion of FAS intravenously or saline once at 24 hours after irradiation. The animals will be euthanized at days 4, 30 or 60 and 6 month following exposure. Proton, and mixed field simulation at total doses of 0.5 Gy or solar particle event (SPE) simulator with total dose of 0.5 Gy over 2 hours will be delivered to the whole bodies of mature adult mice (10/group). We intend to utilize beams at the NASA Space Radiation Laboratory (NSRL), at Brookhaven National Laboratory (BNL). Anti-orthostatic tail suspension will be used to model the unloading, fluid shift, and physiological stress aspects of the microgravity component while the animals were receiving radiation exposure.

A team of specialists will investigate the effects of spaceflight on the response of mice after exposure to proton irradiations. Organ tissues to be investigated include blood, bone, heart, vasculature, skeletal muscle, skin, liver, eye, brain, lung, spleen, pancreas and thymus. For each of these organs, samples will be fixed for analysis leading to identification of pathological changes, as well as changes at the molecular level. Results from the proposed study will address the critical question of impact of space flight or potential synergism between microgravity and radiation. This investigational strategy is unique, integrative, quantitative, and multi-disciplinary and will provide data supporting the benefits of a novel and therapeutic agent (FAS) which is effective in the protection against space conditions of microgravity and irradiation; and the preservation of muscular function essential to operations and activities in space and in outer planet dwelling.

### Results:

Results are pending. However, we expect FAS administration will lead to improved muscle mass, exercise tolerance in space, exercise tolerance during habitation/dwelling in outer planets (including the moon and in Mars.) In addition, we expect mitigation of space (cosmic ray) irradiation harm on the body (on the endothelium and other cells) from the administration of FAS.

EXPERIMENT FIVE: Positive Effects of FAS administration to cancer patients with dysfunctions who are candidates for hematopoietic Cell Transplantation (HCT)

### Introductions:

In a paper published in Transplant Cell Ther 2022 May 17; 28 (8): 498, the authors (Lew et al) mentioned four areas in which cancer patients often showed impairment before treatment: physical, cognitive, nutritional and psychological conditions. They evaluated patients at two time-points: New Patient Evaluation (NPE) and at Sign Off (SO). Their study showed that the Geriatric Assessment (GA) that they used was useful over the entire range of age studied (not just old patients.) This clinical study to be done here will focus on the same kind of patients who are candidates for HCT, but we expect the benefits of FAS administration to be applicable for all cancer (and non-cancer patients with other diseases where improvement in functionality can be beneficial to their treatments and outcomes.)

### Material and Methods:

The biological product (Fibrinoplate-S, FPS) was manufactured according to the US patent application publication dated Dec. 8, 2016, titled “Mass Production of Ready-to-use Suspensions of Fibrinogen-coated Albumin Spheres for the Treatment of Thrombocytopeni Patients” Publication number 20160354481, inventor: Richard C. K. Yen. Patients to be enrolled will be similar in age and dysfunctions according to Lew et al.

After informed consent was signed and the assessment completed at NPE, patients will be divided into two groups: (a) Group A will receive FPS which will be administered intravenously at 2 mL (16 mg) per kg weight of the patient; (b) Group B will receive a placebo of normal saline, also at 2 mL per kg. This dosing regiment has been shown to improve survival from 5% to 60% in mice after a lethal dose of irradiation. The regiment was published in “Fibrinogen-Coated Albumin Nanospheres Prevent Thrombocytopenia-Related Bleeding” authored by Sung et al., published in Radiat Res. 2020 Aug. 1; 194 (2): 162-172. In the mice model, the dose was 1 ml per kg; here we will use 2 mL of FPS per kg; to be administered on day 1, 5, 10 after the day of NPE on day 0. All patients will then be referred to “supportive service” to receive a variety of services including physical therapy.

We will assess: the improvements of patients in all of the four “domains” mentioned in Lew et al. and compare the improvements if any in Group (a) over Group (b).

After HCT we will also assess if Group (a) had improved outcome than in Group (b). Lew et al. admitted that their study has “some limitations” in that they were limited to 2 “pre-HCT time points” in the pre-HCT period and that “future studies should analyze post-HCT function and long-term outcomes” (see Lew's Discussion section.) We intend to include more than 2 time-points in the pre-HCT evaluation because some patients may quit their physical therapy or other recommendations before they can be completed for the SO evaluation. We will also study the side effects and adverse effects of HCT and their long term outcome comparing Group (a) with Group (b).

### Results and Comments:

We expect more patients in Group (a) to show improvement in any of the four domains discussed by Lew et al., than those in Group (b). We also expect less post-HCT side effects from the HCT treatment, and better long-term outcomes in Group (a) compared to Group (b), These results will validate our hypothesis that FPS can result in more stem cells going toward the repair of dysfunctional capacities of the patient, including domains such as generalized physical impairment, cognitive, nutritional (if patient feels better he may eat better) and psychological (all psychological problems can have a physical basis of dysfunction.) Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

What is claimed as being new and desired to be protected by Letters Patent of the United States is as follows:

1. A method of effecting muscle-derived stem cells (MDSCs) or satellite cells (SCs) in a muscle of a subject in need thereof using an albumin nanoparticle suspension containing submicron albumin spheres, the method comprising the step of administering intravenously a therapeutically effective amount of the albumin nanoparticle suspension containing the submicron albumin spheres to the subject, the albumin spheres being configured to suppress pro-inflammatory molecules.

2. The method according to claim 1, wherein the albumin spheres of the albumin nanoparticle suspension are bound with fibrinogen molecules to produce Fibrinogen Albumin Spheres (FAS).

3. The method according to claim 1 further comprising the step of administering to the subject a therapeutically effective amount of one or more co-factor precursors.

4. The method according to claim 3, wherein the co-factor precursors is selected from the group consisting of Nicotinamide Riboside (NR), and Nicotinamide MonoNucleotide (NMN).

5. The method according to claim 1, wherein the effect to the MDSCs or the SCs is an increase in muscle mass in the subject.

6. The method according to claim 1, wherein the effect to the MDSCs or the SCs is a mitigation of microgravity or space radiation-induced damage to the muscle of the subject.

7. The method according to claim 1, wherein the effect to the MDSCs or the SCs is to CD34+ cells residing in the muscle of the subject.

8. The method according to claim 1 further comprising the step of increasing a number of the MDSCs or the SCs in the muscle of the subject by promoting in vivo mobilization of stem cells to the muscle.

9. A method of using an albumin nanoparticle suspension containing submicron albumin spheres to increase muscle-derived stem cells (MDSCs) or satellite cells (SCs) in a muscle of a subject in need thereof, the method comprising the steps of:
a) providing a suspension including fibrinogen-coated albumin nanospheres prepared by coating blank albumin spheres with a solution containing human fibrinogen;
b) administering intravenously a therapeutically effective amount of the albumin nanoparticle suspension to the subject; and
c) increasing a number of the MDSCs or the SCs in the muscle of the subject by promoting in vivo mobilization of stem cells to the muscle to increase muscle mass.

10. The method according to claim 9, wherein the stem cells originate from a location not the muscle.

11. The method according to claim 10, wherein the stem cells originate from bone marrow or from endothelial cells.

12. The method according to claim 9 further comprising the step of administering to the subject a therapeutically effective amount of one or more co-factor precursors.

13. The method according to claim 12, wherein the co-factor precursors is selected from the group consisting of Nicotinamide Riboside (NR), and Nicotinamide MonoNucleotide (NMN).

14. The method according to claim 9, wherein the increase to the number of the MDSCs or the SCs provides a mitigation of microgravity or space radiation-induced damage to the muscle of the subject.

15. The method according to claim 9, wherein the increase to the number of the MDSCs or the SCs is to CD34+ cells residing in the muscle of the subject.
